(19) United States
(12) Patent Application Publication
(10) Pub. No.: US 2005/0281412 A1
(43) Pub. Date: Dec. 22, 2005

(54) Voice prosthesis with neural interface

(76) Inventors: Robert E. Hillman, Weston, MA (US); Steven M. Zeitels, Boston, MA (US); James B. Kobler, Andover, MA (US); James Tracey Heaton, Mansfield, MA (US); Ehab A. Goldstein, Somerville, MA (US)

Correspondence Address: FISH & RICHARDSON PC, P.O. BOX 1022, MINNEAPOLIS, MN, 55440-1022, US

(21) Appl. No.: 10/869,666

(22) Filed: Jun. 16, 2004

(51) Int. Cl. A61F 2/20

(52) U.S. Cl. 381/70; 623/9

(57) ABSTRACT

A voice prosthesis includes a voice actuator for generating a signal to be modulated into speech and a neural interface for receiving a signal indicative of neural activity. A signal processing system in communication with both the neural interface and the voice actuator is configured to provide the voice actuator with a control signal representative of the neural activity.

### FIELD OF INVENTION

The invention relates to prosthetic devices, and in particular, to prosthetic devices for replacing the human voice.

### BACKGROUND

The electrolarynx is an electrically powered device that produces a sound (or buzz) that can be used to acoustically excite the vocal tract as a substitute for laryngeal voice production. The most common electrolarynx is a neck-placed electrolarynx. These electrolarynxes transmit sound energy through neck tissue to provide acoustic excitation of the vocal tract.

Most present electrolarynxes use a simple, battery powered electromechanical driver that operates like a piston hitting a drumhead to produce a “buzz-like” sound. Typically, the patient holds the drumhead against the neck and activates an electromechanical driver. This driver forces a small cylindrical head mounted on a diaphragm to repeatedly strike a rigid plastic disk, thus, producing a series of impulse-like excitations.

In a conventional electrolarynx, the patient must use one hand to hold the device against the neck. The need to use one hand to control the electrolarynx is physically limiting since it precludes normal bimanual function, i.e., performing manual tasks that require the use of both hands while talking.

A conventional electrolarynx also produces speech that sounds non-human (mechanical, robotic, monotone), has reduced intelligibility and loudness, and draws undesirable attention to the user. The poor quality of EL speech has been traced to limitations in performance of current EL sound generating transducers, and to the loss of the fine control of pitch, amplitude, and voice onset and offset timing that is normally provided by the laryngeal mechanism. Loss of fine control causes deficits in voice-related segmental (e.g., voiced-voiceless distinctions for consonants) and suprasegmental (e.g., intonation, syllabic stress) speech parameters.

### SUMMARY

In one aspect, the invention includes a voice prosthesis having a voice actuator for generating a signal to be modulated into speech and a neural interface for receiving a signal indicative of neural activity. A signal processing system in communication with both the neural interface and the voice actuator is configured to provide the voice actuator with a control signal representative of the neural activity.

In some embodiments, the neural interface includes a non-invasive interface. Other embodiments include these in which the neural interface is configured to detect neural activity associated with contraction of neck strap muscles.

Additional embodiments include those in which the voice actuator includes an electrolarynx.

In some embodiments, the signal processing system includes a slow envelope filter for providing a slow envelope signal. For example, the signal processing system can include an oscillator in communication with the slow envelope filter and with the voice actuator. The oscillator is configured to receive the slow envelope signal and to provide a pitch-control signal to the voice actuator. The pitch control signal depends, at least in part, on the slow envelope signal.

Or, the slow envelope filter can be a low-pass filters such filters can include those having a cutoff frequency of 1 Hz.

In other embodiments, the signal processing system includes a fast envelope filter for providing a fast envelope signal. Where a fast-envelope filter is provided, the voice prosthesis can include a switch in communication with the fast envelope filter and with the voice actuator. The switch is configured to actuate the voice actuator at least in part on the basis of the fast envelope signal. One suitable switch is a Schmitt trigger.

In other embodiments of the voice prosthesis, the signal processing system is configured to provide first and second control signals to the voice actuator. The first control signal controls a pitch of the voice actuator in response to the neural activity. The second control signal turns the voice actuator on and off in response to the neural activity.

Embodiments of the voice prosthesis include those in which the signal processing system is an analog signal processing system, as well as those in which the signal processing system is a digital signal processing system.

Another aspect of the invention includes a voice prosthesis having an electrode for detecting a measured signal indicative of neural activity, and first and second low-pass filters. The first low-pass filter has a first cutoff frequency, and an input configured to receive a signal derived from the measured signal. The second low-pass filter has a second cutoff frequency higher than that of the first low-pass filter. Its input is configured to receive the signal derived from the measured signals. An oscillator in communication with an output of the first low-pass filter is configured to provide a drive signal to a voice actuator. The drive signal has a drive frequency that depends in part on an output signal of the first low-pass filter. A switch in communication with an output of the second low-pass filter, is configured to provide an actuating signal to the voice actuator. The actuating signal depends at least in part on an output signal of the second low-pass filter.

In yet another aspect, the invention includes a method for controlling a voice prosthesis by detecting a signal indicative of neural activity; processing that detected signal to obtain a control signal representative of neural activity; and controlling a voice actuator of the voice prosthesis with that control signal.

Specific practices of the method include those in which detecting neural activity includes detecting activity associated with contraction of neck strap muscles, and those in which controlling a voice actuator includes controlling an electro-larynx.

These and other features of the invention will be apparent from the following detailed description and the accompanying figures, in which:

### BRIEF DESCRIPTION OF THE FIGURES

FIG. 1 shows a voice prosthesis;

FIG. 2 shows a signal processing subsystem of the voice prosthesis shown in FIG. 1;

FIG. 3 shows an amplifier and a band pass filter from the signal processing system shown in FIG. 2;

FIG. 4 shows a slow-envelope filter from the signal processing subsystem of FIG. 1;

FIG. 5 shows a fast-envelope filter from the signal processing subsystem of FIG. 1;

FIG. 6 shows typical values for the circuit elements illustrated in FIGS. 3-5; and

FIG. 7 shows a digital implementation of the voice prosthesis of FIG. 1.

### DETAILED DESCRIPTION

When a person attempts to speak, the brain causes nerve impulses to be transmitted to the laryngeal muscles. In a healthy person, these nerve impulses cause appropriate contractions of these muscles. To a great extent, a person modulates the pitch of his voice by modulating the signal that is transmitted to these laryngeal muscles.

A person who has had a laryngectomy has neither his larynx nor his laryngeal muscles available. However, in many cases, the neural signal that would otherwise control the laryngeal muscles or other neck strap muscles related to voice production remains available. The present invention harnesses these neural signals to control a laryngeal prosthesis.

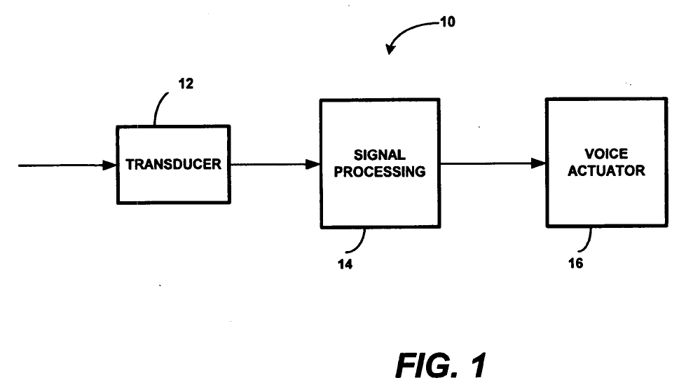

Referring to FIG. 1, a voice prosthesis 10 includes an electro-myographic (“EMG”) transducer 12 in electrical communication with a patient's neck strap muscles for measuring an EMG signal. This measured signal corresponds to the outputs of the nerves that would normally be used for triggering the patient's laryngeal muscles. The output of the transducer 12 is provided to a signal processing system 14 that filters and amplifies the signal. The output of the signal processing system 14 is provided to a voice actuator 16, for example, an electro-larynx, that then generates a mechanical signal. This mechanical signal serves as the foundation for a synthesized voice.

In one embodiment, the voice activator 16 is an electro-larynx having a diaphragm that is placed against the user's neck. A piston striking the diaphragm at a particular driving frequency generates vibrations that travel into the user's trachea. These vibrations enable the user to speak with a synthesized voice tuned to that driving frequency.

The transducer 12 is preferably a non-invasive transducer having an electrode that can be mounted on the surface of the skin. A particularly advantageous location for mounting the electrode is on the surface of the skin covering the neck strap muscles. With the electrode mounted at this location, EMG signals intended to control the laryngeal muscles can readily be measured.

The transducer 12 can also be an invasive transducer, such as a probe, that is placed nearer the source of the EMG signals. In addition, the probe or electrode can be placed at any location at which it can detect EMG or other neural signals intended to control the laryngeal muscles.

A suitable transducer 12 includes a bipolar differential electrode such as that provided by a DE2.1 manufactured by Del Sys Inc. of Boston Mass.

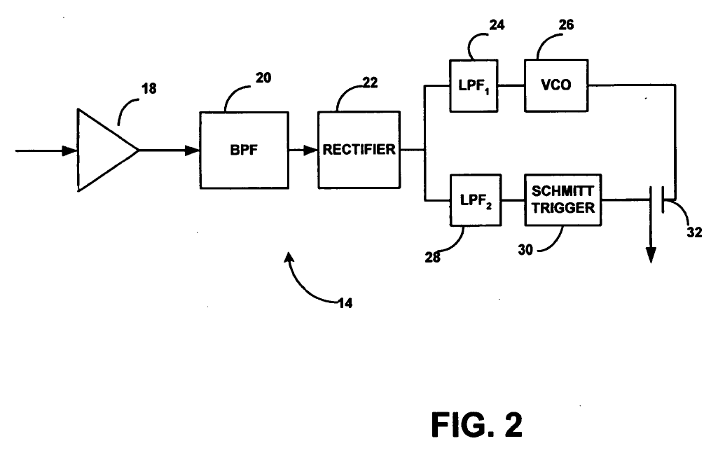

Referring now to FIG. 2, the transducer output signal, which is a signal having an amplitude in the range of tens of millivolts, is provided to an amplifier 18 that amplifies it into an amplified signal having an amplitude in the range of tens of volts. Because most of the energy in the desired EMG signal lies within a particular band of frequencies, the amplified signal is provided to a band-pass filter 20 to reject artifacts and improve signal-to-noise ratio. The desired pass band in this case is between 10 Hz and 500 Hz. To avoid losing energy associated with negative excursions of the signal, the band-pass filtered signal is provided to a rectifier 22.

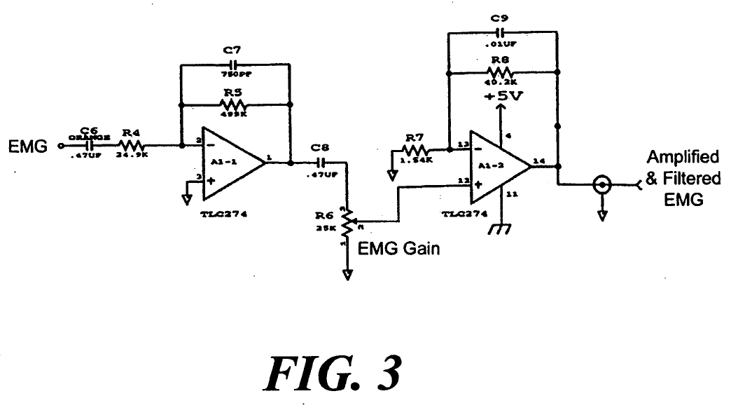

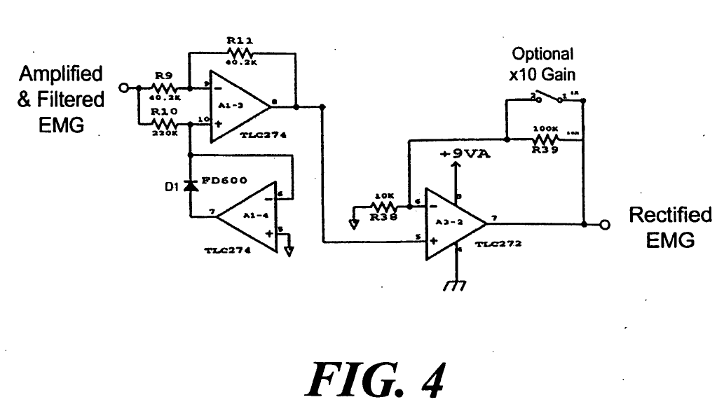

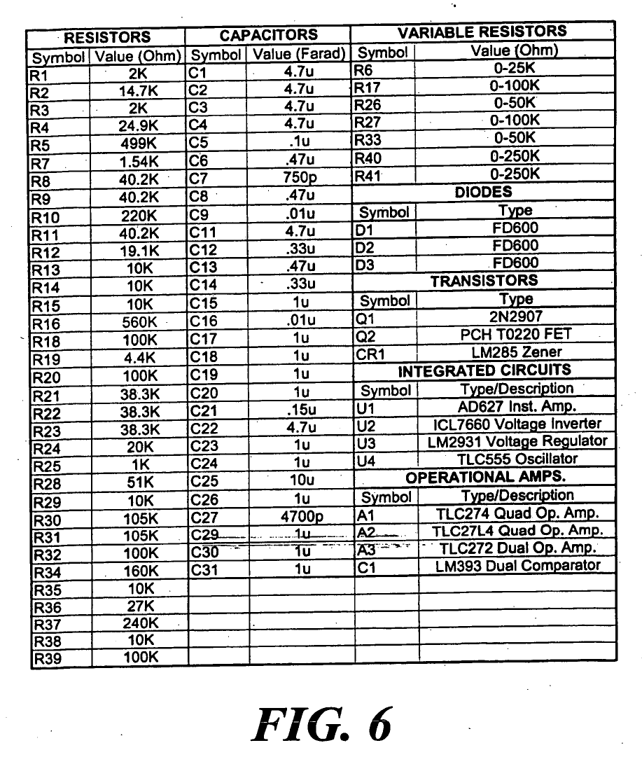

FIG. 3 shows a typical band-pass filter 20 and amplifier 18 for use in the signal processing system. FIG. 4 shows a suitable rectifier 22. Numerical values for circuit parameters in this and subsequent figures are provided in the table of FIG. 6.

As a person speaks, the pitch of that person's voice changes. For example, a rising intonation is often associated with a question. In tonal languages, intonation can completely change the meaning of a phoneme. The frequency with which pitch is modulated in normal speech is, however, much lower than the frequencies that are present in the EMG signal. Even in tonal languages, it is unusual to modulate pitch more than once per spoken word.

If all the frequencies available in the EMG signal were used to modulate the pitch of the voice actuator 16, the result would be a voice having an unpleasant ululating quality, with a pitch that may change several times in the course of a single utterance. For this reason, the rectifier output signal is provided to a slow-envelope low-pass filter 24 (hereafter referred to as the “slow-envelope filter”) having a suitable cut-off frequency. In one embodiment, the slow-envelope filter 24 is a three-pole filter, such as that shown in FIG. 4. However, other types of filters can also be used.

The cut-off frequency of the slow-envelope filter 24 is chosen to be low enough to avoid unpleasant pitch modulation, but high enough to avoid generating a monotonic, or robotic sounding voice. In one embodiment, a cut-off frequency (i.e., 3 dB corner frequency) of 1 Hertz has been found suitable for normal speech.

In the embodiment of FIG. 4, a variable pitch resistor R27 ultimately controls the starting pitch of the user's voice. This is the pitch that the voice actuator 16 produces when the EMG signal has just enough amplitude to turn on the voice actuator 16. To speak with a higher pitch, the user fires additional nerves that would (if such muscles were available) contract additional laryngeal muscles. This increases the EMG signal amplitude, which ultimately causes the pitch of the voice actuator 16 to increase. In the illustrated embodiment, the starting pitch of the voice actuator 16 can be varied by up to 250 Hz.

A pitch slope resistor R26 controls how rapidly the pitch changes with changes in the EMG signal amplitude. A suitable rate of change for normal speech is 112 Hz/volt.

Neither the pitch slope resistor R26 nor the pitch resistor R27 are changed by the user during normal operation. These values are typically adjusted once to customize the prosthesis to the user's neural and voice characteristics.

The slow envelope filter output (hereafter the “slow envelope”) is provided to an input of a voltage-controlled oscillator 26. The resulting output of the voltage-controlled oscillator 26 is a square wave having a fundamental frequency that depends on the amplitude of the slow envelope. This square wave is used to drive the voice actuator 16. In the case of the electro-larynx, the fundamental frequency of the square wave controls the drive frequency at which the piston strikes the diaphragm, and hence the pitch of the synthesized voice. The net effect is that the user controls the pitch of the synthesized voice in much the same way he would have controlled the pitch of his natural voice, namely by controlling the magnitude of the EMG signal of nerves normally used to trigger the laryngeal muscles.

The voltage-controlled oscillator 26 itself has settings to map input frequencies to output frequencies. These settings can be used to control the dynamic range of the synthesized voice. In the case of an analog VCO circuit, the VCO output frequency is a linear function of the input amplitude. However, by replacing the analog VCO circuit with a digital circuit having more complex digital signal processing capabilities, the mapping between input amplitude and output frequency can be altered. This would permit having an oscillator 26 whose output frequency is a non-linear function of the slow envelope amplitude.

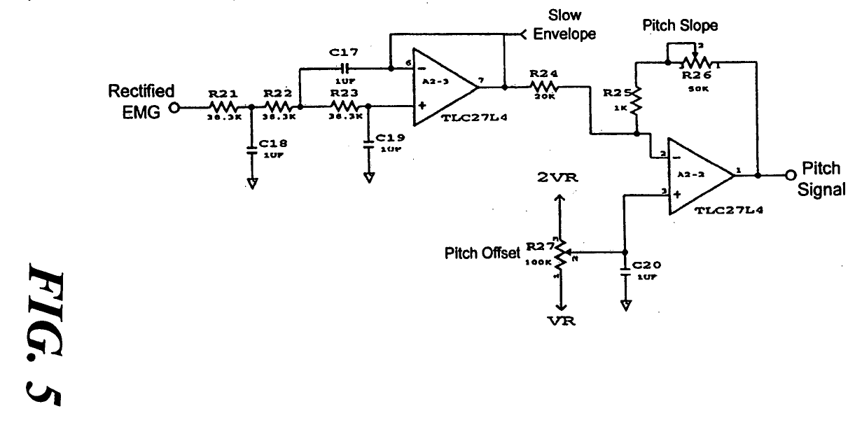

Referring back to FIG. 2, the rectifier output signal is also provided to a fast-envelope low-pass filter 28 (hereafter referred to as the “fast-envelope filter”) having a cut-off frequency that is typically higher than that of the slow-envelope filter. In one embodiment, the fast-envelope filter 28 is a three-pole filter, such as that shown in FIG. 5, having a cut-off frequency between 1 Hz and 9 Hz. The output of the fast-envelope filter 28, referred to as the “fast-envelope,” is used to control a Schmitt trigger 30, also shown in FIG. 5. The Schmitt trigger 30, in turn, controls a switch 32 that turns the voice actuator 16 on and off.

In operation, the user excites nerves that would normally trigger either the laryngeal muscles or any neck-strap muscle used in connection with voice production. When the amplitude of the EMG signal generated by those nerves reaches an upper threshold value, the fast-envelope will have sufficient amplitude to cause the Schmitt trigger 30 to turn on the voice actuator 16. The voice actuator 16 will then produce an output at a frequency consistent with the output of the slow-envelope filter 24. When the EMG signal falls below a lower-threshold value, the output of the fast-envelope filter 28 causes the Schmitt trigger 30 to turn off the voice actuator 16.

The upper threshold value is selected to be low enough such that the user need not exert too much effort to turn on the voice actuator 16, but not so low that the voice actuator 16 is inadvertently turned on. The lower threshold value is selected to be low enough such that the voice actuator 16 does not turn off during low-pitch speech, but not so low that it becomes difficult to turn off the voice actuator 16.

The cutoff frequency of the fast-envelope filter 28 is selected to be low enough such that minor fluctuations in the EMG signal will not cause the voice actuator 16 to turn on and off, but not so low that the voice actuator 16 fails to turn on promptly at the onset of speech. In one embodiment, a cut-off frequency between 1 Hz and 16 Hz has been found to be suitable for many users.

While the embodiments described herein are implemented with analog circuits, it will be apparent that some or all the components can be implemented as digital circuits. Moreover, the components described herein can be incorporated into an integrated circuit, such as an ASIC.

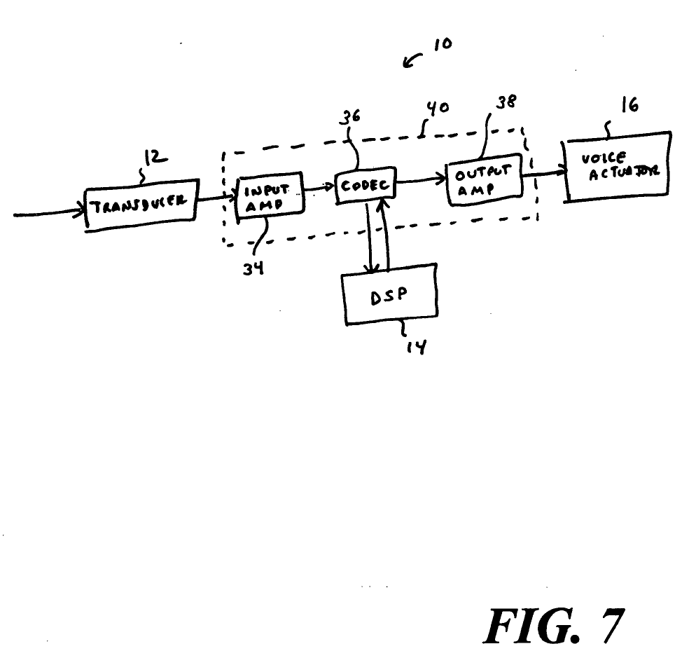

FIG. 7 shows another embodiment in which the signal processing operations shown in FIG. 2 are carried out by a digital signal processor 14. A suitable digital signal processor 14 and associate components is a MOTOROLA DSP56L307 having 3 MB of memory for data and/or program storage, an RS 232 port for communication with a PC, analog input amplifier 34 and output amplifier 38, and a codec 36 for carrying out A/D and D/A conversions. Preferably, the input and output amplifiers 34, 38 are mounted on a replaceable daughter board 40. This results in a system that can easily be made compatible with a variety of analog input and output signals.

Having described the invention, and a preferred embodiment thereof, what we claim as new and secured by letters patent is:

1. A voice prosthesis comprising:
an voice actuator for generating a signal to be modulated into speech;
a neural interface for receiving a signal indicative of neural activity; and
a signal processing system in communication with the neural interface and with the voice actuator, the signal processing system being configured to provide the voice actuator with a control signal representative of the neural activity.

2. The voice prosthesis of claim 1, wherein the neural interface comprises a non-invasive interface.

3. The voice prosthesis of claim 1, wherein the neural interface is configured to detect neural activity associated with contraction of neck strap muscles.

4. The voice prosthesis of claim 1, wherein the voice actuator comprises an electrolarynx.

5. The voice prosthesis of claim 1, wherein the signal processing system comprises a slow envelope filter for providing a slow envelope signal.

6. The voice prosthesis of claim 5, wherein the signal processing system comprises an oscillator in communication with the slow envelope filter and with the voice actuator, the oscillator being configured to receive the slow envelope signal and to provide a pitch-control signal to the voice actuator, the pitch control signal being dependent at least in part on the slow envelope signal.

7. The voice prosthesis of claim 5, wherein the slow envelope filter comprises a low-pass filter.

8. The voice prosthesis of claim 7, wherein the low-pass filter comprises a filter having a cutoff frequency of 1 Hz.

9. The voice prosthesis of claim 1, wherein the signal processing system comprises a fast envelope filter for providing a fast envelope signal.

10. The voice prosthesis of claim 9, further comprising a switch in communication with the fast envelope filter and with the voice actuator, the switch being configured to actuate the voice actuator at least in part on the basis of the fast envelope signal.

11. The voice prosthesis of claim 9, wherein the switch comprises a Schmitt trigger.

12. The voice prosthesis of claim 1, wherein the signal processing system is configured to provide a first and second control signal to the voice actuator, the first control signal controlling a pitch of the voice actuator in response to the neural activity, and the second control signal turning the voice actuator on and off in response to the neural activity.

13. The voice prosthesis of claim 1, wherein the signal processing system comprises an analog signal processing system.

14. The voice prosthesis of claim 1, wherein the signal processing system comprises a digital signal processing system.

15. A voice prosthesis comprising:
an electrode for detecting a measured signal indicative of neural activity;
a first low-pass filter having a first cutoff frequency, the first low-pass filter having an input configured to receiving a signal derived from the measured signal;
a second low-pass filter having a second cutoff frequency that is higher than the first cutoff frequency, the second low-pass filter having an input configured to receive the signal derived from the measured signal;
an oscillator in communication with an output of the first low-pass filter, the oscillator being configured to provide a drive signal to a voice actuator, the drive signal having a drive frequency that depends in part on an output signal of the first low-pass filter; and
a switch in communication with an output of the second low-pass filter, the switch being configured to provide an actuating signal to the voice actuator, the actuating signal depending at least in part on an output signal of the second low-pass filter.

16. A method for controlling a voice prosthesis, the method comprising:
detecting a signal indicative of neural activity;
processing the detected signal to obtain a control signal representative of neural activity; and
controlling a voice actuator of the voice prosthesis with the control signal.

17. The method of claim 16, wherein detecting neural activity comprises detecting activity associated with contraction of neck strap muscles.

18. The method of claim 16, wherein controlling a voice actuator comprises controlling an electro-larynx.
